(19) United States
(12) Patent Application Publication
(10) Pub. No.: US 2011/0172645 A1
(43) Pub. Date: Jul. 14, 2011

(54) WEARABLE DRUG DELIVERY DEVICE INCLUDING INTEGRATED PUMPING AND ACTIVATION ELEMENTS

(75) Inventors: Benjamin J. Moga, Madison, WI (US); Kent B. Chase, Sun Prairie, WI (US); Garrick D.S. Smith, Madison, WI (US)

(73) Assignees: Ratio, Inc.; FluGen, Inc.

(21) Appl. No.: 12/684,832

(22) Filed: Jan. 8, 2010

(51) Int. Cl. A61K 9/22

(52) U.S. Cl. 604/890.1

(57) ABSTRACT

A drug delivery device for delivering a drug to a subject is provided. The drug delivery device includes a housing, a drug reservoir supported by the housing containing the drug and a hollow microneedle supported by the housing. The hollow microneedle is moveable from an inactive position to an activated position, wherein, when the hollow microneedle is moved to the activated position, the tip portion of the hollow microneedle is configured to penetrate the skin of the subject. The drug delivery device includes a channel having an input in communication with the drug reservoir and an output in communication with the hollow microneedle. The channel provides fluid communication between the drug reservoir and the hollow microneedle, such that the drug is permitted to flow from the drug reservoir through the channel and through the hollow microneedle. The channel moves from a first position to a second position as the hollow microneedle moves from the inactive position to the activated position, and the position of the drug reservoir relative to the housing remains fixed as the hollow microneedle moves from the inactive position to the activated position.

### BACKGROUND

The present invention relates generally to the field of drug delivery devices. The present invention relates specifically to wearable active transdermal drug delivery devices including integrated pumping and activation elements to facilitate drug delivery using a microneedle as the point of drug delivery.

An active agent or drug (e.g., pharmaceuticals, vaccines, hormones, nutrients, etc.) may be administered to a patient through various means. For example, a drug may be ingested, inhaled, injected, delivered intravenously, etc. In some applications, a drug may be administered transdermally. In some transdermal applications, such as transdermal nicotine or birth control patches, a drug is absorbed through the skin. Passive transdermal patches often include an absorbent layer or membrane that is placed on the outer layer of the skin. The membrane typically contains a dose of a drug that is allowed to be absorbed through the skin to deliver the substance to the patient. Typically, only drugs that are readily absorbed through the outer layer of the skin may be delivered with such devices.

Other drug delivery devices are configured to provide for increased skin permeability to the delivered drugs. For example, some devices use a structure, such as one or more microneedles, to facilitate transfer of the drug into the skin. Solid microneedles may be coated with a dry drug substance. The puncture of the skin by the solid microneedles increases permeability of the skin allowing for absorption of the drug substance. Hollow microneedles may be used to provide a fluid channel for drug delivery below the outer layer of the skin. Other active transdermal devices utilize other mechanisms (e.g., iontophoresis, sonophoresis, etc.) to increase skin permeability to facilitate drug delivery.

### SUMMARY

One embodiment of the invention relates to a drug delivery device for delivering a drug to a subject. The drug delivery device includes a housing, a drug reservoir supported by the housing, the drug reservoir containing the drug, and a hollow microneedle supported by the housing. The hollow microneedle is moveable from an inactive position to an activated position, wherein, when the hollow microneedle is moved to the activated position, the tip portion of the hollow microneedle is configured to penetrate the skin of the subject. The drug delivery device includes a channel having an input in communication with the drug reservoir and an output in communication with the hollow microneedle. The input of the channel is in fluid communication with the drug reservoir when the hollow microneedle is in the inactive position. The channel provides fluid communication between the drug reservoir and the hollow microneedle, such that the drug is permitted to flow from the drug reservoir through the channel and through the hollow microneedle. The channel moves from a first position to a second position as the hollow microneedle moves from the inactive position to the activated position, and the position of the drug reservoir relative to the housing remains fixed as the hollow microneedle moves from the inactive position to the activated position.

Another embodiment of the invention relates to a device for delivering a liquid drug into the skin of a subject. The device includes a housing, a drug reservoir coupled to the housing, a conduit coupled to and integral with the reservoir, a microneedle coupled to the conduit and a microneedle actuator coupled to the microneedle. The microneedle actuator is located within the housing and is configured impart kinetic energy to the microneedle to drive the microneedle into the skin of the subject upon activation.

Another embodiment of the invention relates to a wearable drug delivery device for delivering a liquid drug into the skin of a subject. The device includes a housing, an attachment element for attaching the drug delivery device to the skin of the subject, a drug reservoir for storing a dose of the liquid drug supported by the housing and a microneedle array including a plurality of hollow microneedles. Each of the hollow microneedles includes a tip portion and a central channel extending through the tip portion. The microneedle array moveable from an inactive position to an activated position, wherein, when the microneedle array is moved to the activated position, the tip portions of the hollow microneedles are configured to penetrate the skin of the subject. The device includes a drug channel extending from the drug reservoir and coupled to the microneedle array such that the drug reservoir is in fluid communication with the tip portions of the hollow microneedles and a channel arm extending between the drug reservoir and the microneedle array. The drug channel is formed at least in part of the material of the channel arm, and the channel arm comprises a flexible material that bends as the channel arm is moved from a first position to a second position as the hollow microneedle array moves from the inactive position to the activated position. The channel arm is integral with the drug reservoir. The device includes a microneedle attachment element coupling the microneedle array to the channel arm in both the inactive position and the active position and a microneedle actuator comprising stored energy. The microneedle actuator located within the housing and configured to transfer the stored energy to the microneedle component to cause the microneedle component to move from the inactive position to the activated position.

Alternative exemplary embodiments relate to other features and combinations of features as may be generally recited in the claims

### BRIEF DESCRIPTION OF THE FIGURES

This application will become more fully understood from the following detailed description, taken in conjunction with the accompanying figures, wherein like reference numerals refer to like elements in which:

FIG. 1 is a perspective view of a drug delivery device assembly having a cover and a protective membrane according to an exemplary embodiment;

FIG. 2 is a perspective view of a drug delivery device according to an exemplary embodiment after both the cover and protective membrane have been removed;

FIG. 3 is a exploded perspective view of a drug delivery device assembly according to an exemplary embodiment;

FIG. 4 is a exploded perspective view of a drug delivery device showing various components mounted within the device housing according to an exemplary embodiment;

FIG. 5 is a exploded perspective view of a drug delivery device showing various components removed from the device housing according to an exemplary embodiment;

FIG. 6 is a perspective sectional view showing a drug delivery device prior to activation according to an exemplary embodiment;

FIG. 7 is a perspective sectional view showing a drug delivery device following activation according to an exemplary embodiment;

FIG. 8 is a side sectional view showing a drug delivery device following activation according to an exemplary embodiment;

FIG. 9 is a side sectional view showing a drug delivery device following delivery of a drug according to an exemplary embodiment;

FIG. 10 is a side sectional view showing a drug delivery device prior to activation according to an exemplary embodiment;

FIG. 11 is a side sectional view showing a drug delivery device indicating movement of the device components during activation according to an exemplary embodiment;

FIG. 12 is a side sectional view showing a drug delivery device following activation indicating activity of the pumping system and drug delivery flow path according to an exemplary embodiment; and

FIG. 13 is an enlarged sectional view showing a portion of a drug delivery device following activation indicating the drug delivery flow path through a microneedle component according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring generally to the figures, a substance delivery device assembly is shown according to various exemplary embodiments. The delivery device assembly includes various packaging and/or protective elements that provide for protection during storage and transportation. The assembly also includes a substance delivery device that is placed in contact with the skin of a subject (e.g., a human or animal, etc.) prior to delivery of the substance to the subject. After the device is affixed to the skin of the subject, the device is activated in order to deliver the substance to the subject. Following delivery of the substance, the device is removed from the skin.

The delivery device described herein may be utilized to deliver any substance that may be desired. In one embodiment, the substance to be delivered is a drug, and the delivery device is a drug delivery device configured to deliver the drug to a subject. As used herein the term “drug” is intended to include any substance delivered to a subject for any therapeutic, preventative or medicinal purpose (e.g., vaccines, pharmaceuticals, nutrients, nutraceuticals, etc.). In one such embodiment, the drug delivery device is a vaccine delivery device configured to deliver a dose of vaccine to a subject. In one embodiment, the delivery device is configured to deliver a flu vaccine. The embodiments discussed herein relate primarily to a device configured to deliver a substance intradermally. In other embodiments, the device may be configured to deliver a substance transdermally or may be configured to deliver drugs directly to an organ other than the skin.

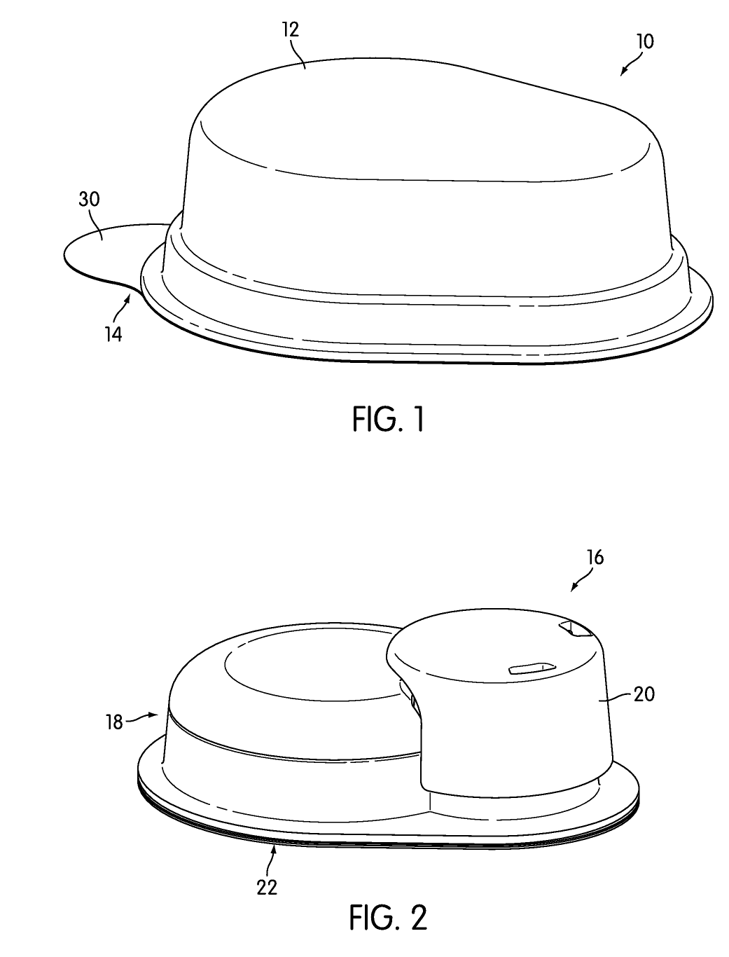

Referring to FIG. 1, drug delivery device assembly 10 is depicted according to an exemplary embodiment. Drug delivery device assembly 10 includes an outer protective cover 12 and a protective membrane or barrier 14 that provides a sterile seal for drug delivery device assembly 10. As shown in FIG. 1, drug delivery device assembly 10 is shown with cover 12 and protective barrier 14 in an assembled configuration. Generally, cover 12 and protective barrier 14 protect various components of drug delivery device 16 during storage and transport prior to use by the end user. In various embodiments, cover 12 may be made of a relatively rigid material (e.g., plastic, metal, cardboard, etc.) suitable to protect other components of drug delivery device assembly 10 during storage or shipment. As shown, cover 12 is made from a non-transparent material. However, in other embodiments cover 12 is a transparent or semi-transparent material.

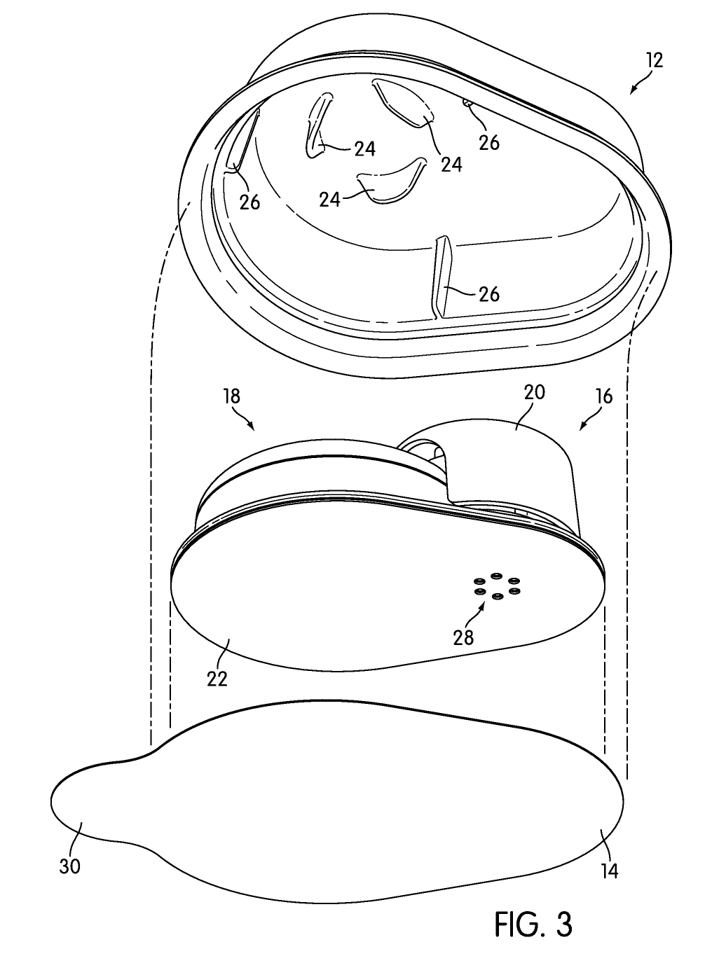

As shown in FIG. 2 and FIG. 3, the drug delivery device assembly includes delivery device 16. Delivery device 16 includes a housing 18, an activation control, shown as, but not limited to, button 20, and an attachment element, shown as, but not limited to, adhesive layer 22. Adhesive layer 22 includes one or more holes 28 (see FIG. 3). Holes 28 provide a passageway for one or more hollow drug delivery microneedles as discussed in more detail below. During storage and transport, cover 12 is mounted to housing 18 of delivery device 16 such that delivery device 16 is received within cover 12. In the embodiment shown, cover 12 includes three projections or tabs 24 extending from the inner surface of the top wall of cover 12 and three projections or tabs 26 extending from the inner surface of the sidewall of cover 12. When cover 12 is mounted to delivery device 16, tabs 24 and 26 contact the outer surface of housing 18 such that delivery device 16 is positioned properly and held within cover 12. Protective barrier 14 is attached to the lower portion of cover 12 covering adhesive layer 22 and holes 28 during storage and shipment. Together, cover 12 and protective barrier 14 act to provide a sterile and hermetically sealed packaging for delivery device 16.

Referring to FIG. 3, to use delivery device 16 to deliver a drug to a subject, protective barrier 14 is removed exposing adhesive layer 22. In the embodiment shown, protective barrier 14 includes a tab 30 that facilitates griping of protective barrier 14 during removal. Once adhesive layer 22 is exposed, delivery device 16 is placed on the skin. Adhesive layer 22 is made from an adhesive material that forms a nonpermanent bond with the skin of sufficient strength to hold delivery device 16 in place on the skin of the subject during use. Cover 12 is released from delivery device 16 exposing housing 18 and button 20 by squeezing the sides of cover 12. With delivery device 16 adhered to the skin of the subject, button 20 is pressed to trigger delivery of the drug to the patient. When delivery of the drug is complete, delivery device 16 may be detached from the skin of the subject by applying sufficient force to overcome the grip generated by adhesive layer 22.

In one embodiment, delivery device 16 is sized to be conveniently wearable by the user during drug delivery. In one embodiment, the length of delivery device 16 along the device's long axis is 53.3 mm, the length of delivery device 16 along the device's short axis (at its widest dimension) is 48 mm, and the height of delivery device 16 at button 20 following activation is 14.7 mm. However, in other embodiments other dimensions are suitable for a wearable drug delivery device. For example, in another embodiment, the length of delivery device 16 along the device's long axis is between 40 mm and 80 mm, the length of delivery device 16 along the device's short axis (at its widest dimension) is between 30 mm and 60 mm, and the height of delivery device 16 at button 20 following activation is between 5 mm and 30 mm. In another embodiment, the length of delivery device 16 along the device's long axis is between 50 mm and 55 mm, the length of delivery device 16 along the device's short axis (at its widest dimension) is between 45 mm and 50 mm, and the height of delivery device 16 at button 20 following activation is between 10 mm and 20 mm.

While in the embodiments shown the attachment element is shown as, but not limited to, adhesive layer 22, other attachment elements may be used. For example, in one embodiment, delivery device 16 may be attached via an elastic strap. In another embodiment, delivery device 16 may not include an attachment element and may be manually held in place during delivery of the drug. Further, while the activation control is shown as button 20, the activation control may be a switch, trigger, or other similar element, or may be more than one button, switch, trigger, etc., that allows the user to trigger delivery of the drug.

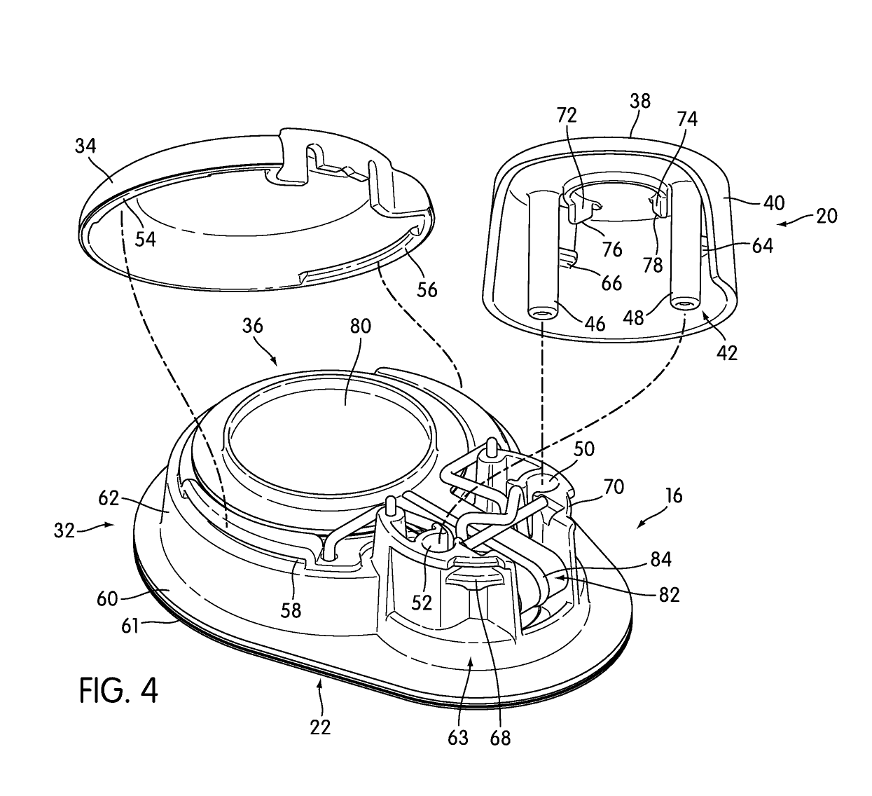

Referring to FIG. 4, housing 18 of delivery device 16 includes a base portion 32 and a reservoir cover 34. Base portion 32 includes a flange 60, a bottom tensile member, shown as bottom wall 61, a first support portion 62 and a second support portion 63. In the embodiment shown, bottom wall 61 is a rigid wall that is positioned below flange 60. As shown in FIG. 4, the outer surface of first support portion 62 is generally cylindrically shaped and extends upward from flange 60. Second support portion 63 is generally cylindrically shaped and extends upward from flange 60 to a height above first support portion 62. As shown in FIG. 4, delivery device 16 includes a substance delivery assembly 36 mounted within base portion 32 of housing 18.

Reservoir cover 34 includes a pair of tabs 54 and 56 that each extend inwardly from a portion of the inner edge of cover 34. Base portion 32 includes a recess 58 and second recess similar to recess 58 on the opposite side of base portion 32. As shown in FIG. 4, both recess 58 and the opposing recess are formed in the upper peripheral edge of the outer surface of first support portion 62. When reservoir cover 34 is mounted to base portion 32, tab 54 is received within recess 58 and tab 56 is received within the similar recess on the other side of base portion 32 to hold cover 34 to base portion 32.

As shown in FIG. 4, button 20 includes a top wall 38. Button 20 also includes a sidewall or skirt 40 that extends from a portion of the peripheral edge of top wall 38 such that skirt 40 defines an open segment 42. Button 20 is shaped to receive the generally cylindrical shaped second support portion 63 of base portion 32. Button 20 includes a first mounting post 46 and a second mounting post 48 both extending in a generally perpendicular direction from the lower surface of top wall 38. Second support portion 63 includes a first channel 50 and a second channel 52. Mounting posts 46 and 48 are slidably received within channels 50 and 52, respectively, when button 20 is mounted to second support portion 63. Mounting posts 46 and 48 and channels 50 and 52 act as a vertical movement guide for button 20 to help ensure that button 20 moves in a generally downward vertical direction in response to a downward force applied to top wall 38 during activation of delivery device 16. Precise downward movement of button 20 ensures button 20 interacts as intended with the necessary components of substance delivery assembly 36 during activation.

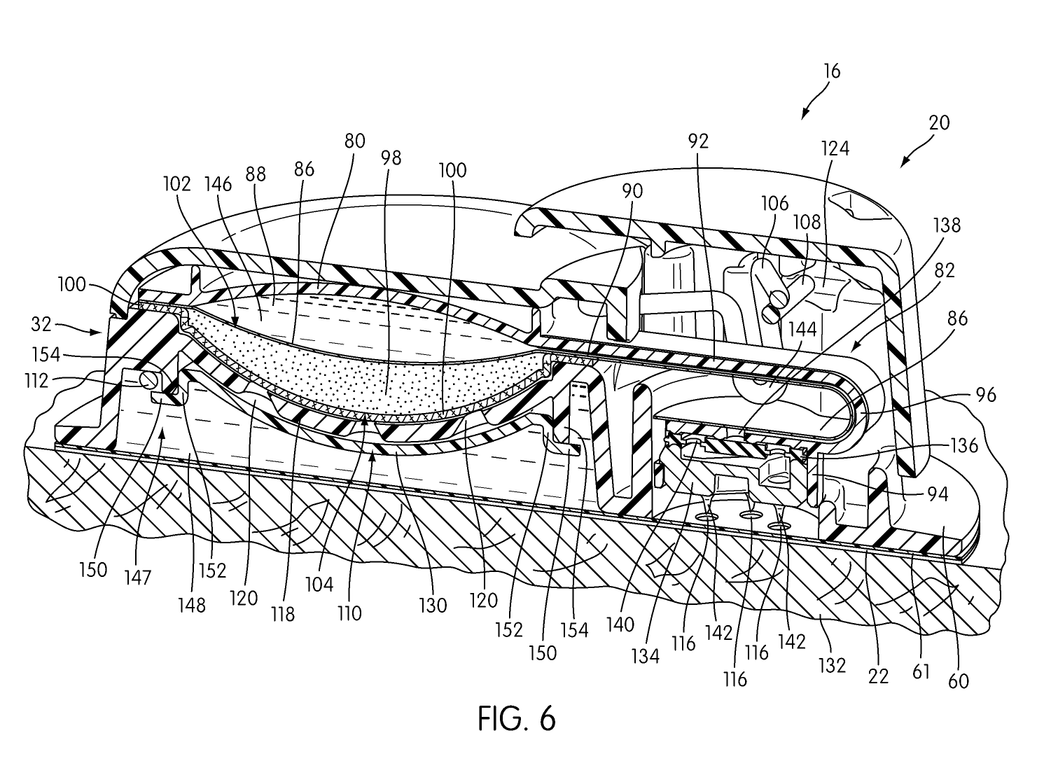

Button 20 also includes a first support ledge 64 and a second support ledge 66 both extending generally perpendicular to the inner surface of sidewall 40. The outer surface of second support portion 63 includes a first button support surface 68 and second button support surface 70. When button 20 is mounted to second support portion 63, first support ledge 64 engages and is supported by first button support surface 68 and second support ledge 66 engages and is supported by second button support surface 70. The engagement between ledge 64 and surface 68 and between ledge 66 and surface 70 supports button 20 in the pre-activation position (shown for example in FIG. 6). Button 20 also includes a first latch engagement element 72 and a second latch engagement element 74 both extending in a generally perpendicular direction from the lower surface of top wall 38. First latch engagement element 72 includes an angled engagement surface 76 and second latch engagement element 74 includes an angled engagement surface 78.

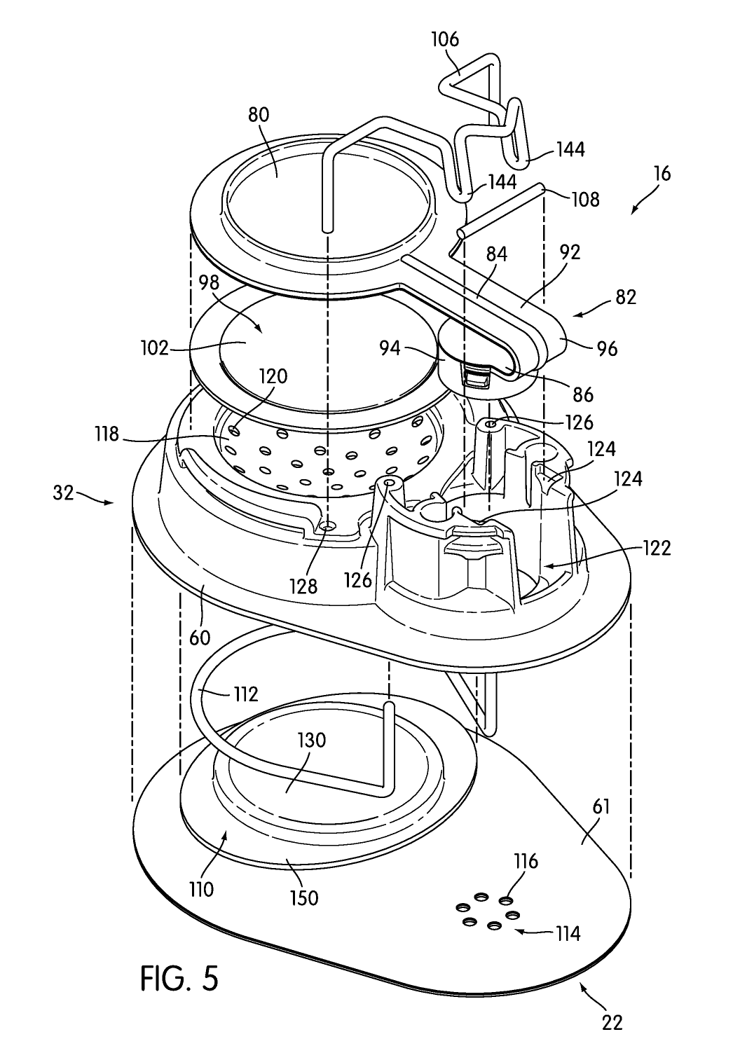

Referring to FIG. 4 and FIG. 5, substance delivery assembly 36 includes a drug reservoir base 80 and drug channel arm 82. The lower surface of drug channel arm 82 includes a depression or groove 84 that extends from reservoir base 80 along the length of drug channel arm 82. As shown in FIG. 4 and FIG. 5, groove 84 appears as a rib protruding from the upper surface of drug channel arm 82. Substance delivery assembly 36 further includes a flexible barrier film 86 adhered to the inner surfaces of both drug reservoir base 80 and drug channel arm 82. Barrier film 86 is adhered to form a fluid tight seal or a hermetic seal with drug reservoir base 80 and channel arm 82. In this arrangement (shown best in FIGS. 6-9), the inner surface of drug reservoir base 80 and the inner surface of barrier film 86 form a drug reservoir 88, and the inner surface of groove 84 and the inner surface of barrier film 86 form a fluid channel, shown as, but not limited to, drug channel 90. In this embodiment, drug channel arm 82 acts as a conduit to allow fluid to flow from drug reservoir 88. As shown, drug channel arm 82 includes a first portion 92 extending from drug reservoir base 80, a microneedle attachment portion, shown as, but not limited to, cup portion 94, and a generally U-shaped portion 96 joining the first portion 92 to the cup portion 94. In the embodiment shown, drug reservoir base 80 and drug channel arm 82 are made from an integral piece of polypropylene. However, in other embodiments, drug reservoir base 80 and drug channel arm 82 may be separate pieces joined together and may be made from other plastics or other materials.

Substance delivery assembly 36 includes a reservoir actuator or force generating element, shown as, but not limited to, hydrogel 98, and a fluid distribution element, shown as, but not limited to, wick 100 in FIG. 6. Because FIG. 5 depicts delivery device 16 in the pre-activated position, hydrogel 98 is formed as a hydrogel disc and includes a concave upper surface 102 and a convex lower surface 104. As shown, wick 100 is positioned below hydrogel 98 and is shaped to generally conform to the convex shape of lower surface 104.

Substance delivery assembly 36 includes a microneedle activation element or microneedle actuator, shown as, but not limited to, torsion rod 106, and a latch element, shown as, but not limited to, latch bar 108. As explained in greater detail below, torsion rod 106 stores energy, which upon activation of delivery device 16, is transferred to one or more microneedles causing the microneedles to penetrate the skin. Substance delivery assembly 36 also includes a fluid reservoir plug 110 and plug disengagement bar 112. Bottom wall 61 is shown removed from base portion 32, and adhesive layer 22 is shown coupled to the lower surface of bottom wall 61. Bottom wall 61 includes one or more holes 114 that are sized and positioned to align with holes 28 in adhesive layer 22. In this manner, holes 114 in bottom wall 61 and holes 28 in adhesive layer 22 form channels, shown as needle channels 116.

As shown in FIG. 5, first support portion 62 includes a support wall 118 that includes a plurality of fluid channels 120. When assembled, wick 100 and hydrogel 98 are positioned on support wall 118 below drug reservoir 88. As shown, support wall 118 includes an upper concave surface that generally conforms to the convex lower surfaces of wick 100 and hydrogel 98. Fluid reservoir plug 110 includes a concave central portion 130 that is shaped to generally conform to the convex lower surface of support wall 118. First support portion 62 also includes a pair of channels 128 that receive the downwardly extending segments of torsion rod 106 such that the downwardly extending segments of torsion rod 106 bear against the upper surface of bottom wall 61 when delivery device 16 is assembled. Second support portion 63 includes a central cavity 122 that receives cup portion 94, U-shaped portion 96 and a portion of first portion 92 of drug channel arm 82. Second support portion 63 also includes a pair of horizontal support surfaces 124 that support latch bar 108 and a pair of channels 126 that slidably receive the vertically oriented portions of plug disengagement bar 112.

Referring to FIG. 6, a perspective, sectional view of delivery device 16 is shown attached or adhered to skin 132 of a subject prior to activation of the device. As shown, adhesive layer 22 provides for gross attachment of the device to skin 132 of the subject. Delivery device 16 includes a microneedle component, shown as, but not limited to, microneedle array 134, having a plurality of microneedles, shown as, but not limited to, hollow microneedles 142, extending from the lower surface of microneedle array 134. In the embodiment shown, microneedle array 134 includes an internal channel 141 allowing fluid communication from the upper surface of microneedle array 134 to the tips of hollow microneedles 142. Delivery device 16 also includes a valve component, shown as, but not limited to, check valve 136. Both microneedle array 134 and check valve 136 are mounted within cup portion 94. Drug channel 90 terminates in an aperture or hole 138 positioned above check valve 136. In the pre-activation or inactive position shown in FIG. 6, check valve 136 blocks hole 138 at the end of drug channel 90 preventing a substance, shown as, but not limited to, drug 146, within drug reservoir 88 from flowing into microneedle array 134. While the embodiments discussed herein relate to a drug delivery device that utilizes hollow microneedles, in other various embodiments, other microneedles, such as solid microneedles, may be utilized.

As shown in FIG. 6, in the pre-activation position, latch bar 108 is supported by horizontal support surfaces 124. Latch bar 108 in turn supports torsion rod 106 and holds torsion rod 106 in the torqued, energy storage position shown in FIG. 6. Torsion rod 106 includes a U-shaped contact portion 144 that bears against a portion of the upper surface of barrier film 86 located above cup portion 94. In another embodiment, U-shaped contact portion 144 is spaced above barrier film 86 (i.e., not in contact with barrier film 86) in the pre-activated position.

Delivery device 16 includes an activation fluid reservoir, shown as, but not limited to, fluid reservoir 147, that contains an activation fluid, shown as, but not limited to, water 148. In the embodiment shown, fluid reservoir 147 is positioned generally below hydrogel 98. In the pre-activation position of FIG. 6, fluid reservoir plug 110 acts as a plug to prevent water 148 from flowing from fluid reservoir 147 to hydrogel 98. In the embodiment show, reservoir plug 110 includes a generally horizontally positioned flange 150 that extends around the periphery of plug 110. Reservoir plug 110 also includes a sealing segment 152 that extends generally perpendicular to and vertically away from flange 150. Sealing segment 152 of plug 110 extends between and joins flange 150 with the concave central portion 130 of plug 110. The inner surface of base portion 32 includes a downwardly extending annular sealing segment 154. The outer surfaces of sealing segment 152 and/or a portion of flange 150 abut or engage the inner surface of annular sealing segment 154 to form a fluid-tight seal preventing water from flowing from fluid reservoir 147 to hydrogel 98 prior to device activation.

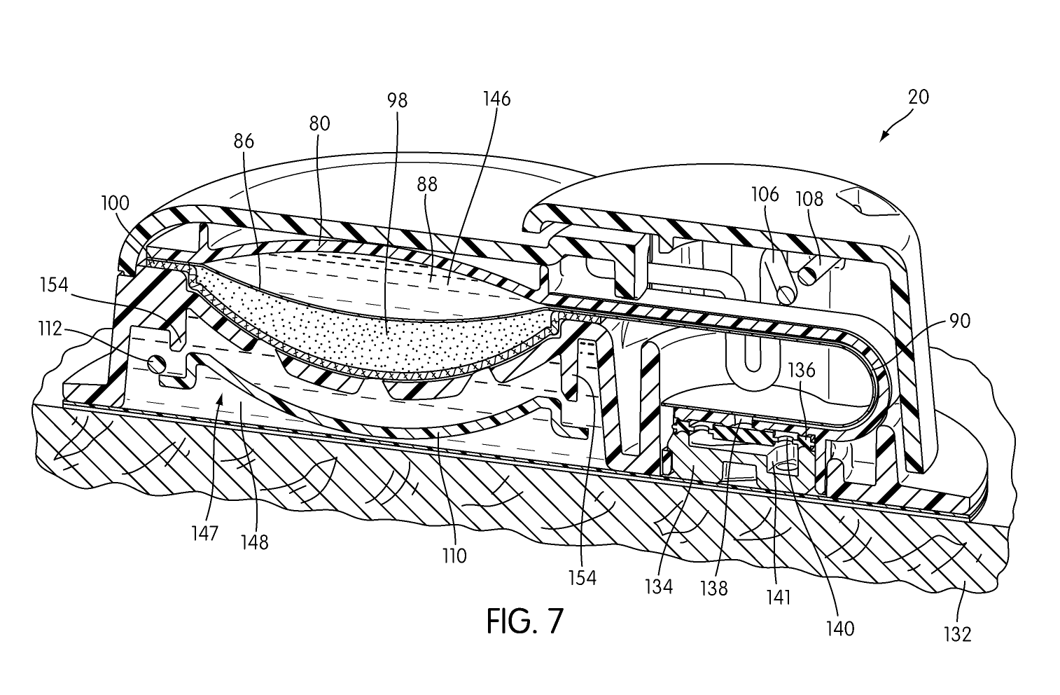

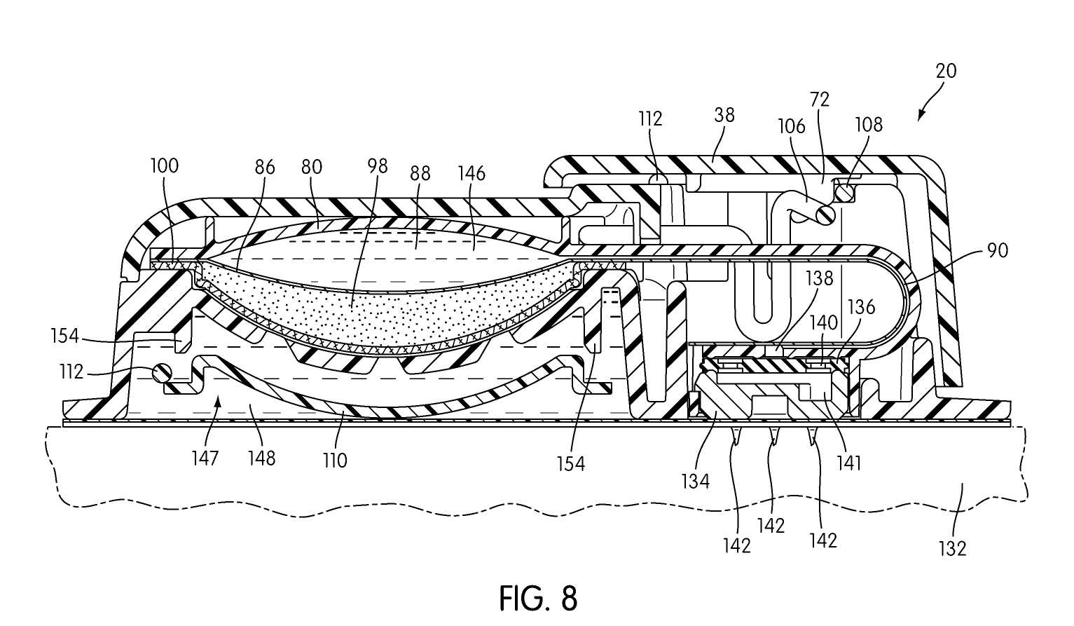

Referring to FIG. 7 and FIG. 8, delivery device 16 is shown immediately following activation. In FIG. 8, skin 132 is drawn in broken lines to show hollow microneedles 142 after insertion into the skin of the subject. To activate delivery device 16, button 20 is pressed in a downward direction (toward the skin). Movement of button 20 from the pre-activation position of FIG. 6 to the activated position causes activation of both microneedle array 134 and of hydrogel 98. Depressing button 20 causes first latch engagement element 72 and second latch engagement element 74 to engage latch bar 108 and to force latch bar 108 to move from beneath torsion rod 106 allowing torsion rod 106 to rotate from the torqued position of FIG. 6 to the seated position of FIG. 7. The rotation of torsion rod 106 drives microneedle array 134 downward and causes hollow microneedles 142 to pierce skin 132. In addition, depressing button 20 causes the lower surface of button top wall 38 to engage plug disengagement bar 112 forcing plug disengagement bar 112 to move downward. As plug disengagement bar 112 is moved downward, fluid reservoir plug 110 is moved downward breaking the seal between annular sealing segment 154 of base portion 32 and sealing segment 152 of reservoir plug 110.

With the seal broken, water 148 within reservoir 147 is put into fluid communication with hydrogel 98. As water 148 is absorbed by hydrogel 98, hydrogel 98 expands pushing barrier film 86 upward toward drug reservoir base 80. As barrier film 86 is pushed upward by the expansion of hydrogel 98, pressure within drug reservoir 88 and drug channel 90 increases. When the fluid pressure within drug reservoir 88 and drug channel 90 reaches a threshold, check valve 136 is forced open allowing drug 146 within drug reservoir 88 to flow through aperture 138 at the end of drug channel 90. As shown, check valve 136 includes a plurality of holes 140, and microneedle array 134 includes a plurality of hollow microneedles 142. Drug channel 90, hole 138, plurality of holes 140 of check valve 136, internal channel 141 of microneedle array 134 and hollow microneedles 142 define a fluid channel between drug reservoir 88 and the subject when check valve 136 is opened. Thus, drug 146 is delivered from reservoir 88 through drug channel 90 and out of the holes in the tips of hollow microneedles 142 to the skin of the subject by the pressure generated by the expansion of hydrogel 98.

In the embodiment shown, check valve 136 is a segment of flexible material (e.g., medical grade silicon) that flexes away from aperture 138 when the fluid pressure within drug channel 90 reaches a threshold placing drug channel 90 in fluid communication with hollow microneedles 142. In one embodiment, the pressure threshold needed to open check valve 136 is about 0.5-1.0 pounds per squire inch (psi). In various other embodiments, check valve 136 may be a rupture valve, a swing check valve, a ball check valve, or other type of valve the allows fluid to flow in one direction. In the embodiment shown, the microneedle actuator is a torsion rod 106 that stores energy for activation of the microneedle array until the activation control, shown as button 20, is pressed. In other embodiments, other energy storage or force generating components may be used to activate the microneedle component. For example, in various embodiments, the microneedle activation element may be a coiled compression spring or a leaf spring. In other embodiments, the microneedle component may be activated by a piston moved by compressed air or fluid. Further, in yet another embodiment, the microneedle activation element may be an electromechanical element, such as a motor, operative to push the microneedle component into the skin of the patient.

In the embodiment shown, the actuator that provides the pumping action for drug 146 is a hydrogel 98 that expands when allowed to absorb water 148. In other embodiments, hydrogel 98 may be an expandable substance that expands in response to other substances or to changes in condition (e.g., heating, cooling, pH, etc.). Further, the particular type of hydrogel utilized may be selected to control the delivery parameters. In various other embodiments, the actuator may be any other component suitable for generating pressure within a drug reservoir to pump a drug in the skin of a subject. In one exemplary embodiment, the actuator may be a spring or plurality of springs that when released push on barrier film 86 to generate the pumping action. In another embodiment, the actuator may be a manual pump (i.e., a user manually applies a force to generate the pumping action). In yet another embodiment, the actuator may be an electronic pump.

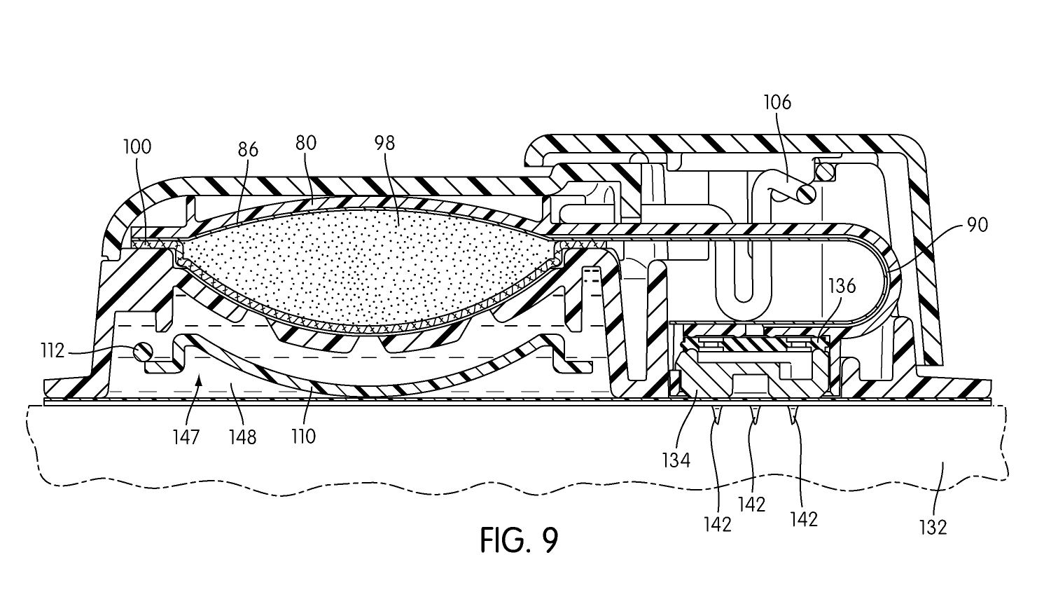

Referring to FIG. 9, delivery device 16 is shown following completion of delivery of drug 146 to the subject. In FIG. 9, skin 132 is drawn in broken lines. As shown in FIG. 9, hydrogel 98 expands until barrier film 86 is pressed against the lower surface of reservoir base 80. When hydrogel 98 has completed expansion, substantially all of drug 146 has been pushed from drug reservoir 88 into drug channel 90 and delivered to skin 132 of the subject. The volume of drug 146 remaining within delivery device 16 (i.e., the dead volume) following complete expansion by hydrogel 98 is minimized by configuring the shape of drug reservoir 88 to enable complete evacuation of the drug reservoir and by minimizing the volume of fluid pathway formed by drug channel 90, hole 138, plurality of holes 140 of check valve 136 and hollow microneedles 142. In the embodiment shown, delivery device 16 is a single-use, disposable device that is detached from skin 132 of the subject and is discarded when drug delivery is complete. However, in other embodiments, delivery device 16 may be reusable and is configured to be refilled with new drug, to have the hydrogel replaced, and/or to have the microneedles replaced.

In one embodiment, delivery device 16 and reservoir 88 are sized to deliver a dose of drug of up to approximately 500 microliters. In other embodiments, delivery device 16 and reservoir 88 are sized to allow delivery of other volumes of drug (e.g., up to 200 microliters, up to 400 microliters, up to 1 milliliter, etc.).

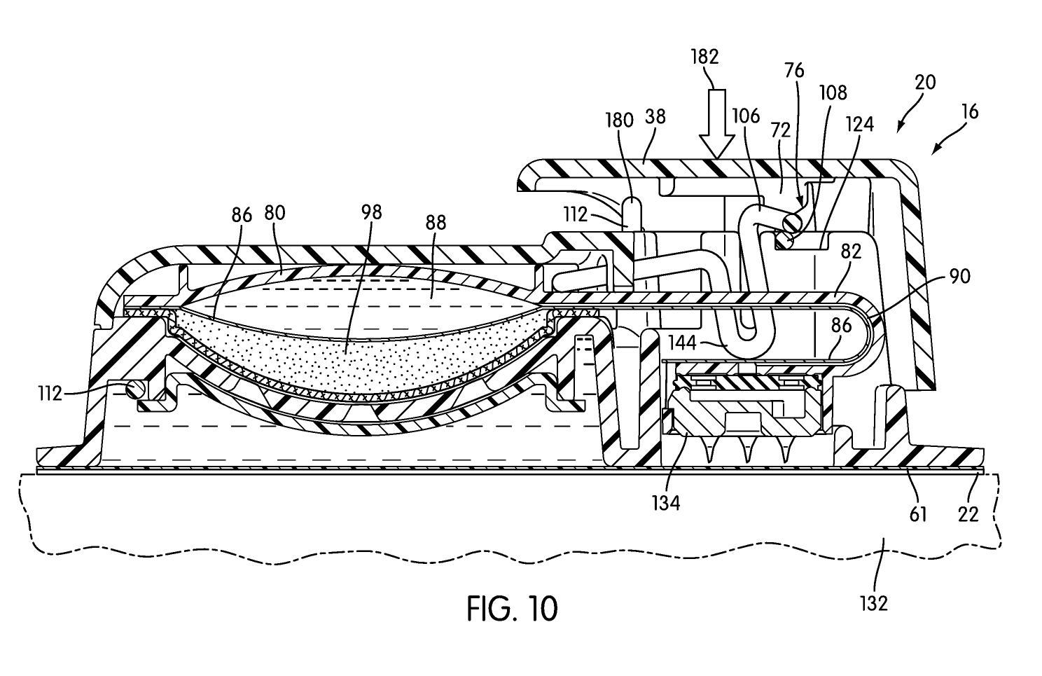

Referring generally to FIGS. 10-13, drug delivery device 16 is shown in greater detail and includes features that provide a wearable, compact drug delivery device with integrated pumping and activation elements. FIG. 10 shows a side sectional view of delivery device 16 in the pre-activated or inactive position. The microneedle activation element or microneedle actuator, shown as torsion rod 106, is shown supported by a latch element, shown as latch bar 108. Latch bar 108 is supported by horizontal support surface 124. In the pre-activated position, latch bar 108 is positioned at the rear of horizontal support surface 124 (i.e., the part of horizontal support surface closest to reservoir 88) to engage and support torsion rod 106. Further, in the inactive position, first latch engagement element 72 extends from the lower surface of top wall 38 of button 20. In this position, angled engagement surface 76 of first latch engagement element 72 is positioned directly above latch bar 108. U-shaped contact portion 144 of torsion bar 106 is in contact with barrier film 86 and poised above microneedle array 134. In another embodiment, U-shaped contact portion 144 is spaced above barrier film 86 (i.e., not in contact with barrier film 86) in the pre-activated position. Plug disengagement bar 112 includes a button engagement portion 180 that extends upwardly from channels 126 (shown in FIG. 5) in base portion 32. In the inactive position the lower surface of top wall 38 of button 20 is positioned above button engagement portion 180 of plug disengagement bar 112. As discussed above, drug channel arm 82 extends from drug reservoir base 80 and barrier film 86 is adhered to both reservoir base 80 and drug channel arm 82 to form drug reservoir 88 and drug channel 90. Microneedle array 134 is mounted within cup portion 94 of drug channel arm 82. In the embodiment shown, drug channel arm 82 is rigid enough to support or hold microneedle array 134 above bottom wall 61 in the inactive position.

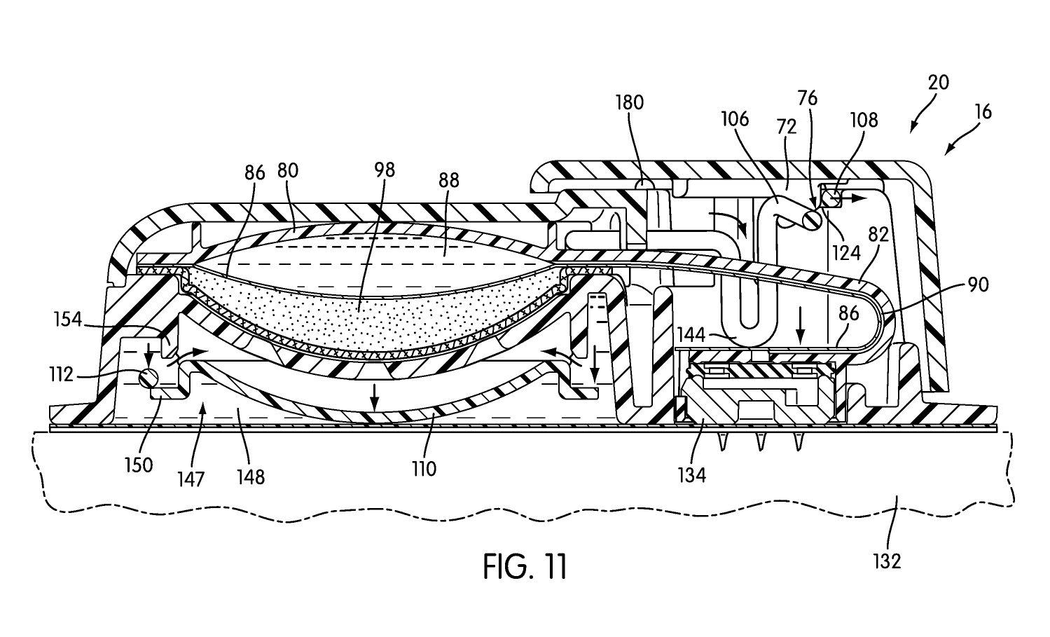

The microneedle activation element or microneedle actuator, shown as, but not limited to, torsion rod 106, stores potential energy that is released upon depression of button 20. In this embodiment, the energy used to move microneedle array 134 from the inactive to the active position is stored by torsion rod 106 completely within housing 18. Thus, the energy used to move microneedle array 134 from the inactive to the active position does not need to be supplied to delivery device 16 from an external source. To activate drug delivery device 16, a downward force 182 is applied to button 20. FIG. 11 depicts delivery device 16 following activation with arrows indicating movement of various parts triggered by depression of button 20. As button 20 moves downward, angled engagement surface 76 of first latch engagement element 72 engages latch bar 108. As first latch engagement element 72 moves downward, latch bar 108 is pushed to the right along horizontal support surface 124 such that torsion rod 106 is released. When released, torsion rod 106 twists clockwise (in the view of FIG. 11) bearing against the upper surface of barrier film 86 above microneedle array 134. The release of the energy stored in torsion rod 106 forces microneedle array 134 downward to cause hollow microneedles 142 to pierce skin 132 of the subject.

In the embodiment shown, torsion rod 106 includes two U-shaped contact portions 144 (see FIG. 5). The two U-shaped contact portions 144 of torsion rod 106 straddle drug channel 90 and engage barrier film 86 above the lateral edges of microneedle array 134. This configuration allows contact between U-shaped contact portions 144 and barrier film 86 while preventing U-shaped contact portions 144 from closing or compressing drug channel 90.

In other embodiments, the microneedle actuator may be a coiled compression spring or a leaf spring. However, torsion rod 106 provides a compact actuator that this is suited for a wearable embodiment of delivery device 16. Torsion rod 106 is configured to store more energy within a smaller space than some other force generation components, such as compression springs and leaf springs. Further, as can be seen in FIGS. 10 and 11, as torsion rod 106 moves from the inactive to active position, the height of torsion rod 106 relative to housing 18 decreases.

Delivery device 16 is also configured to allow microneedle array 134 to move from the inactive to the active position while remaining in fluid communication with drug reservoir 88 and drug channel 90. Because microneedle array 134 is mounted within cup portion 94 of drug channel arm 82, drug channel arm 82 must be able to move along with microneedle array 134 while drug reservoir 88 remains in place. In the embodiment shown in FIG. 10, drug channel arm 82 is made from a flexible material such that drug channel arm 82 is allowed to bend, flex, or move with microneedle array 134 as microneedle array 134 is moved from the inactive position to the active position. As shown best in FIG. 11, flexing of drug channel arm 82 along its length allows microneedle array 134 to move downward to engage skin 132 without occluding or collapsing drug channel 90. The flexibility of drug channel arm 82 allows drug channel arm 82 to be integral with drug reservoir base 80 while allowing the position of drug reservoir base 80 relative to housing 18 to remain fixed during activation.

Further referring to FIG. 10, in addition to triggering the release of torsion rod 106 and activation of microneedle array 134, depression of button 20 also triggers the start of drug delivery by activating the actuator or force generating element, shown as, but not limited to, hydrogel 98. Depression of button 20 brings the lower surface of top wall 38 of button 20 into engagement with button engagement portion 180 of plug disengagement bar 112. Because plug disengagement bar 112 is rigid, the downward movement of button engagement portion 180 caused by depression of button 20 causes plug disengagement bar 112 to move downward. As shown in FIG. 11, as plug disengagement bar 112 moves downward, disengagement bar 112 engages flange 150 of reservoir plug 110 causing reservoir plug to disengage from annular sealing segment 154.

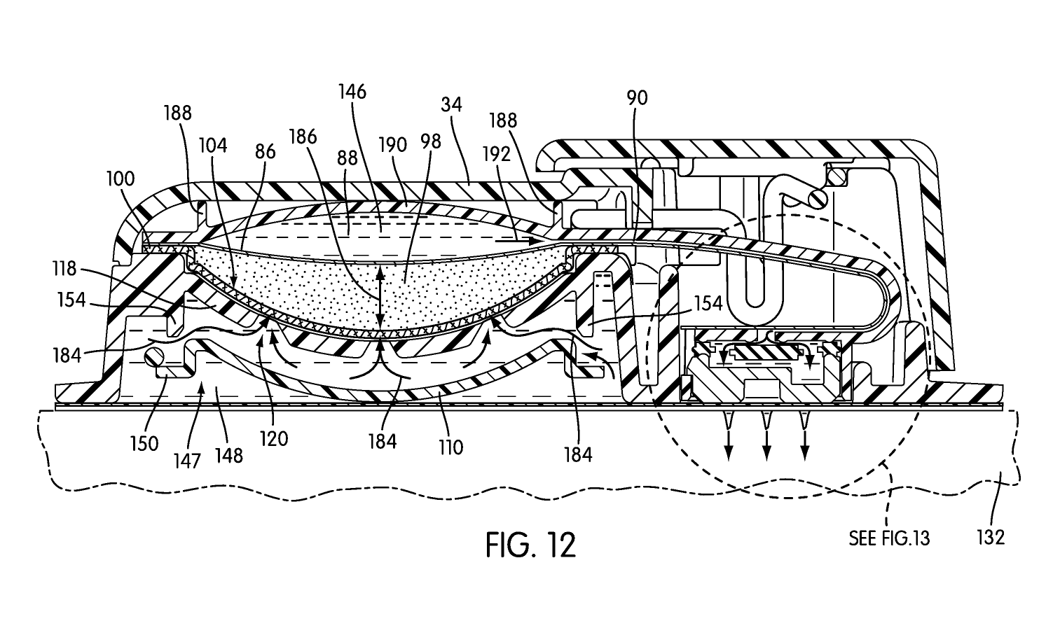

After disengagement of reservoir plug 110 from annular sealing segment 154, reservoir plug 110 is moved to the bottom of fluid reservoir 147 as shown in FIG. 12. With reservoir plug released from annular sealing segment 154, water 148 in fluid reservoir 147 is placed into fluid communication with hydrogel 98. As depicted by arrows 184, water 148 is permitted to flow from fluid reservoir 147 to wick 100 through channels 120 formed in support wall 118. Wick 100 absorbs water 148 and transmits it to hydrogel 98. In one embodiment, wick 100 is made of a hydrophilic material. As hydrogel 98 absorbs water 148, hydrogel 98 expands as indicated by arrow 186. As discussed above, wick 100 is shaped to match the convex lower surface 104 of hydrogel 98, and thus, wick 100 is in contact with the substantially the entire lower surface 104 of hydrogel 98. This arrangement allows wick 100 to evenly distribute water 148 to hydrogel 98 to facilitate even expansion of hydrogel 98. In addition, wick 100 acts as a barrier preventing hydrogel 98 from expanding into and blocking channels 120 in support wall 118.

Further referring to FIG. 12, as hydrogel 98 expands, it pushes on the portion of barrier film 86 below drug reservoir 88 increasing the pressure within drug reservoir 88 and within drug channel 90. Reservoir base 80 is rigidly supported such that expansion of hydrogel 98 is able to generate pressure to force drug 146 from the reservoir through drug channel 90 and into skin 132 of the subject. The pressure within drug reservoir 88 generated by expansion of hydrogel 98 would be less if reservoir base 80 were allowed to deform as hydrogel 98 expands.

As shown in FIG. 12, to further resist deformation of reservoir base 80, the outer surface of the central portion 190 of reservoir base 80 is in contact with the lower surface of reservoir cover 34. Further, reservoir base 80 includes an annular rim or collar 188 extending upwardly from and generally perpendicular to the upper surface of reservoir base 80. Collar 188 contacts the lower surface of reservoir cover 34 resisting deformation of reservoir base 80 that may otherwise be caused by expansion of hydrogel 98. In the embodiment shown, collar 188 is positioned toward the peripheral edge of reservoir base 80 such that collar 188 provides support along the peripheral edge of reservoir base 80 and central portion 190 provides support in the center of reservoir base 80. In addition to providing resistance to deformation, the contact between central portion 190 and collar 188 of reservoir base 80 and the lower surface of reservoir cover 34 provides for a tight assembly within housing 18.

Support wall 118 is also constructed of a rigid material to facilitate pressure generation within drug reservoir 88 by expansion of hydrogel 98. In other words, support wall 118 provides a rigid surface for hydrogel 98 to push against during expansion. The material of wick 100 and the size of fluid channels 120 in support wall 118 are selected to provide sufficient support for hydrogel 98 during expansion.

In the embodiment shown, drug channel arm 82 and drug reservoir base 80 are made from an integral piece of material, such as polypropylene. In this embodiment, as shown in FIG. 12, the thickness of the material of drug channel arm 82 is generally the same as the thickness of the material of drug reservoir base 80. In this embodiment, the thickness of the material of drug channel arm 82 and drug reservoir base 80 is such that drug channel arm 82 is permitted to bend during activation. In this embodiment, the rigidity of drug reservoir base 80 is supplied primarily by the support provided by collar 188, the contact between the outer surface of central portion 190 and the lower surface of reservoir cover 34, and the circular domed-shape of drug reservoir base 80. In another embodiment, drug channel arm 82 and drug reservoir base 80 may be made from an integral piece of material with varying thickness. In one such embodiment, the thickness of the material of drug channel arm 82 may be less than the thickness of the material of drug reservoir base 80. In this embodiment, the greater thickness of the material in drug reservoir base 80 may provide for sufficient rigidity without other support structures, while the smaller thickness of the drug channel arm 82 allows drug channel arm 82 to bend. In yet another embodiment, drug reservoir base 80 may be made from a rigid material, and drug channel arm 82 may be made from a different, flexible material.

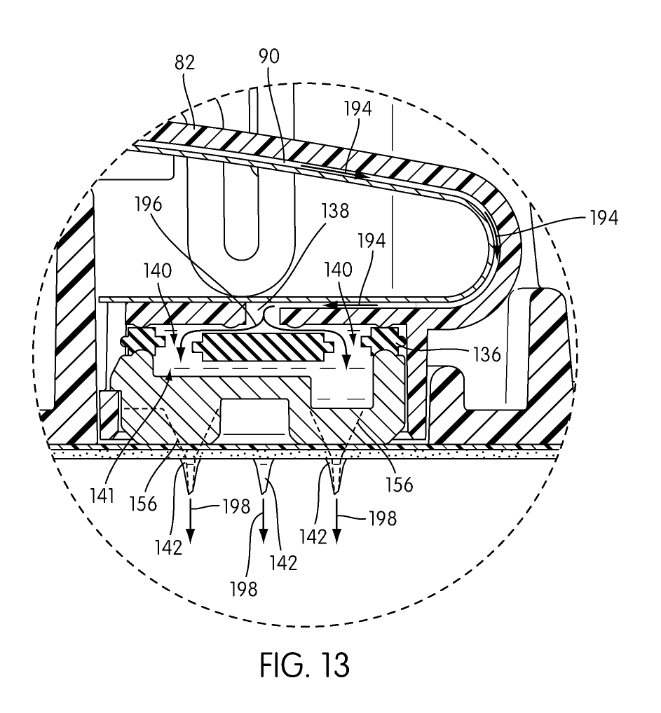

As hydrogel 98 expands, drug 146 is pushed from drug reservoir 88 and into drug channel 90 as indicated by arrow 192. As shown in FIG. 13, drug 146 flows through drug channel 90 to aperture 138 as indicated by arrows 194. When pressure within drug channel 90 reaches the threshold discussed above, check valve 136 flexes away from aperture 138 allowing drug 146 to flow through aperture 138. As indicated by arrows 196, drug 146 then flows through holes 140 in check valve 136 and into internal channel 141 of microneedle array 134. Drug 146 then flows through internal channel 141 through central channels 156 of hollow microneedles 142 to be delivered to skin 132 of the subject as indicated by arrows 198.

Further modifications and alternative embodiments of various aspects of the invention will be apparent to those skilled in the art in view of this description. Accordingly, this description is to be construed as illustrative only. The construction and arrangements of the drug delivery device assembly and the drug delivery device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. A drug delivery device for delivering a drug to a subject, the device comprising:
a housing;
a drug reservoir supported by the housing, the drug reservoir containing the drug;
a hollow microneedle supported by the housing, the hollow microneedle moveable from an inactive position to an activated position, wherein, when the hollow microneedle is moved to the activated position, the tip portion of the hollow microneedle is configured to penetrate the skin of the subject; and
a channel having an input in communication with the drug reservoir and an output in communication with the hollow microneedle, wherein the input of the channel is in fluid communication with the drug reservoir when the hollow microneedle is in the inactive position, wherein the channel provides fluid communication between the drug reservoir and the hollow microneedle, such that the drug is permitted to flow from the drug reservoir through the channel and through the hollow microneedle;
wherein the channel moves from a first position to a second position as the hollow microneedle moves from the inactive position to the activated position, and further wherein the position of the drug reservoir relative to the housing remains fixed as the hollow microneedle moves from the inactive position to the activated position.

2. The device of claim 1, further comprising a channel arm extending between the drug reservoir and the hollow microneedle, the channel formed at least in part of the material of the channel arm, wherein the channel arm is integral with the drug reservoir.

3. The device of claim 2, wherein the channel arm comprises a flexible material and the channel arm bends as the channel is moved from the first position to the second position.

4. The device of claim 2, wherein the channel arm includes a microneedle attachment portion coupled to the hollow microneedle in both the inactive position and the activated position.

5. The device of claim 2, further comprising a reservoir base and a flexible film coupled to the reservoir base such that an inner surface of the reservoir base and an inner surface of the flexible film define the drug reservoir.

6. The device of claim 5, wherein the channel arm includes a depression running the length of the channel arm, wherein the flexible film is coupled to the channel arm such that an inner surface of the depression and the inner surface of the flexible film define the channel.

7. The device of claim 6, further comprising a reservoir actuator in contact with the flexible film, the reservoir actuator configured to increase pressure within the drug reservoir to move the drug from the drug reservoir through the channel and through the tips of the hollow microneedle to deliver the drug to the skin of the subject.

8. The device of claim 7, wherein the reservoir actuator is a hydrogel configured to expand when placed in contact with an activation fluid.

9. The device of claim 8, further comprising an activation fluid reservoir and a fluid distribution element positioned between the activation fluid reservoir and the hydrogel.

10. The device of claim 9, wherein the activation fluid is water and the fluid distribution element is a hydrophilic wick configured to transmit water from the activation fluid reservoir to the hydrogel.

11. The device of claim 1, further comprising a microneedle actuator comprising stored energy, the microneedle actuator located within the housing and configured to release stored energy to cause the hollow microneedle to move from the inactive position to the activated position.

12. The device of claim 11, wherein the microneedle actuator is a torsion rod.

13. A device for delivering a liquid drug into the skin of a subject, the device comprising:
a housing;
a drug reservoir coupled to the housing;
a conduit coupled to and integral with the drug reservoir;
a microneedle coupled to the conduit; and
a microneedle actuator located within the housing, wherein the microneedle actuator is configured to impart kinetic energy to the microneedle to drive the microneedle into the skin of the subject upon activation.

14. The device of claim 13, further comprising an activation control moveable from a first position to a second position to cause activation of the microneedle.

15. The device of claim 14, wherein the microneedle actuator is a torsion rod.

16. The device of claim 15, wherein the torsion rod is supported by a latch bar when the activation control is in the first position.

17. The device of claim 16, wherein movement of the activation control from the first position to the second position moves the latch bar to release the torsion rod.

18. The device of claim 17, wherein the activation control is a button, the button including a top wall and at least one engagement surface extending from the lower surface of the top wall, wherein as the button is moved from the first position to the second position the engagement surface engages the latch bar to release the torsion rod.

19. A wearable drug delivery device for delivering a liquid drug into the skin of a subject, the device comprising:
a housing;
an attachment element for attaching the drug delivery device to the skin of the subject;
a drug reservoir for storing a dose of the liquid drug, the drug reservoir supported by the housing;
a microneedle array including a plurality of hollow microneedles, each of the hollow microneedles including a tip portion and a central channel extending through the tip portion, the microneedle array moveable from an inactive position to an activated position, wherein, when the microneedle array is moved to the activated position, the tip portions of the hollow microneedles are configured to penetrate the skin of the subject;
a drug channel extending from the drug reservoir and coupled to the microneedle array such that the drug reservoir is in fluid communication with the tip portions of the hollow microneedles;
a channel arm extending between the drug reservoir and the microneedle array, the drug channel formed at least in part of the material of the channel arm, wherein the channel arm comprises a flexible material and the channel arm bends as the channel arm is moved from a first position to a second position as the microneedle array moves from the inactive position to the activated position, and further wherein the channel arm is integral with the drug reservoir;
a microneedle attachment element coupling the microneedle array to the channel arm in both the inactive position and the activated positions; and
a microneedle actuator comprising stored energy, the microneedle actuator located within the housing, the microneedle actuator configured to transfer the stored energy to the microneedle array to cause the microneedle array to move from the inactive position to the activated position.

20. The device of claim 19, wherein the microneedle actuator is a torsion rod.
